(19) United States
(12) Patent Application Publication
(10) Pub. No.: US 2003/0143738 A1
(43) Pub. Date: Jul. 31, 2003

(54) Compositions and methods for treating ischemic stroke

(76) Inventors: Hiroki Yokota, Carmel, ID (US); Hui Bin Sun, Indianapolis, IN (US); Zao C. Xu, Carmel, IN (US); Yiwen Ruan, Indianapolis, IN (US)

Correspondence Address: DANN DORFMAN HERRELL & SKILLMAN, SUITE 720, 1601 MARKET STREET, PHILADELPHIA, PA, 19103-2307, US

(21) Appl. No.: 10/291,046

(22) Filed: Nov. 8, 2002

Related U.S. Application Data
(60) Provisional application No. 60/336,980, filed on Nov. 8, 2001.

(51) Int. Cl. A61K 48/00; C12Q 1/68; C07H 21/04; C12N 5/00

(52) U.S. Cl. 435/375; 514/44; 536/23.5; 435/6

(57) ABSTRACT

Compositions and methods are provided for the treatment of ischemic stroke.

### CROSS-REFERENCE TO RELATED APPLICATION

This application claims priority to U.S. Provisional Application No. 60/336,980, filed Nov. 8, 2001, the entire disclosure being incorporated by reference herein.

Pursuant to 35 U.S.C. §202(c), it is acknowledged that the U.S. Government has certain rights in the invention described herein, which was made in part with funds from the National Institute of Health, Grant Nos. AHA0070048 and NS38053.

### FIELD OF THE INVENTION

The present invention describes compositions and methods for the treatment of stroke. More specifically, antisense oligonucleotides are provided which inhibit neurodegeneration associated with cerebral ischemia.

### BACKGROUND OF THE INVENTION

Several publications and patent documents are referenced in this application by numerals in parentheses in order to more fully describe the state of the art to which this invention pertains. Full citations for these references are found at the end of the specification. The disclosure of each of these publications and patent documents is incorporated by reference herein.

Approximately 500,000 Americans each year suffer a stroke and nearly 150,000 die from the event or its ensuing complications. To date, more than 300,000 people in the United States are experiencing serious long term disability as the result of a stroke. Such disabilities may be neurological or functional and include paralysis, aphasia, vision loss, memory deficits and personality changes. As a result, as many as 30% of stroke survivors require some assistance to perform the normal activities associated with daily living.

Ischemic strokes account for approximately 80% of all strokes, and are caused by acute interruption of arterial blood flow to the brain by a thrombus or embolic blockage. Hemorrhagic strokes are caused by blood vessel rupture and bleeding into the sub-arachnoid or intracerebral areas of the brain.

The interruption of blood flow to any region of the brain gives rise to a complex series of deleterious cellular metabolic events. Immediately following a cerebral ischemic attack, delivery of oxygen and glucose to brain cells is compromised. This results in regional brain dysfunction which becomes apparent as neuronal activity begins to fail. If neuronal cells remain structurally intact and the ionic gradients remain undisturbed, this mild-to-moderate form of ischemia is often reversible when treated immediately after initial stroke symptoms materialize. However, if oxygen deprivation persists, pronounced cellular and neurological dysfunction will ensue. Sodium and chloride ions rapidly accumulate within cells, accompanied by an inflow of water, and cytotoxic edema causes rapid swelling of the neurons and glia. The level of calcium ions inside the cells may also rise dramatically which leads to irreversible cellular injury.

There are at least two general therapeutic approaches for the treatment of stroke. The first approach targets the shortfall of available arterial oxygen and glucose relative to the needs of local brain tissue by enhancing blood flow to the brain. In ischemic stroke, one can lyse an arterial thrombus within a few hours after symptom onset using tissue plasminogen activator (tPA), a thrombolytic factor. However, a major drawback to this treatment is that the tPA must be administered within three (3) hours after the initial stroke symptoms develop. Unfortunately, only 1% to 2% of stroke patients actually meet this criteria for treatment.

The second therapeutic approach, neuroprotection, aims to reduce the intrinsic vulnerability of brain tissue to ischemia. Neuroprotective approaches have focused mainly on blocking excitotoxicity, i.e., neuronal cell death triggered by the excitatory transmitter, glutamate, and mediated by cytotoxic levels of calcium influx. Potential neuroprotective compounds include glutamate-receptor antagonists and blockers of voltage-gated sodium or calcium channels which attenuate excitotoxicity. To date, clinical trials using the glutamate-receptor antagonist, N-methyl-D-aspartate (NMDA), to improve the survival of neuronal cells have not been encouraging (6). One reason for the lack of success using these types of neuroprotective compounds is a delay in neuronal cell death after ischemic injury. During ischemia, there is a sharp initial increase in the extracellular concentration of glutamate which returns to normal levels after 30 minute reperfusion (7). However, a few days pass before neurons actually begin to degenerate.

Thus, a need exists for improved compositions and methods to alleviate or prevent strokes and the complications resulting therefrom.

### SUMMARY OF THE INVENTION

In accordance with the present invention, antisense molecules targeted to nucleic acids encoding calcium-independent receptor α-latrotoxin (CIRL) are provided. The antisense molecules of the invention specifically hybridize with nucleic acid molecules encoding CIRL and inhibit the expression CIRL. In a preferred embodiment, the antisense molecules of the invention are oligonucleotides comprising the sequences of SEQ ID NO: 7 and SEQ ID NO: 8. The antisense molecules provided above may optionally comprise modified phosphodiester backbones which enhance in vivo stability. Phosphorothioates represent an exemplary modified phosphodiester backbone.

According to another aspect of the invention, a method is provided for inhibiting the in vivo expression of CIRL in hippocampal cells or tissues. The method comprises contacting hippocampal cells or tissues in vivo with an antisense molecule of the invention so that expression of CIRL is inhibited.

In a related aspect of the invention, a method for inhibiting the expression of CIRL in human cells is provided. The method comprises providing an antisense molecule comprising the sequence of SEQ ID NO: 7 or SEQ ID NO: 8, which hybridizes to an expression-controlling sequence of a nucleic acid molecule encoding CIRL and administering the antisense oligonucleotide to human cells under conditions whereby the antisense oligonucleotide enters the cells and binds specifically to the expression-controlling sequence of the nucleic acid molecule encoding CIRL in an amount sufficient to inhibit expression of CIRL.

In another embodiment of the present invention, a method is provided for blocking the neurodegeneration of hippocampal CA1 neuronal cells caused by ischemic stroke. The method comprises delivering an antisense molecule comprising the sequence of SEQ ID NO: 7 or SEQ ID NO: 8 to hippocampal cells which binds specifically to the nucleic acid molecule encoding CIRL in an amount sufficient to inhibit expression of CIRL.

In yet another embodiment of the invention, a pharmaceutical preparation is provided for treating ischemic stroke. The pharmaceutical preparation includes an antisense oligonucleotide comprising the sequence of SEQ ID NO: 7 or SEQ ID NO: 8 in a biologically compatible medium. In yet another aspect, the pharmaceutical preparation may optionally comprise at least one targeting agent for improving delivery of the antisense molecule to hippocampal cells.

In another aspect of the invention, antibodies immunologically specific for CIRL are provided. Such antibodies may be monoclonal or polyclonal, and include recombinant, chimerized, humanized, antigen binding fragments of such antibodies, and anti-idiotypic antibodies.

In another aspect of the invention, methods for detecting the expression of CIRL-associated molecules in a biological sample are provided. Such molecules include CIRL encoding DNA, RNA, or fragments thereof, and CIRL proteins and fragments thereof. Exemplary methods comprise mRNA analysis, for example by RT-PCR, and immunological methods, for example contacting a sample with a detectably labeled antibody immunologically specific for CIRL protein, and determining the presence of CIRL expression as a function of the amount of detectably labeled antibody bound by the sample relative to control cells. In a preferred embodiment, these assays may be used in diagnostic tests for ischemic stroke damage, and to assess compounds which might alleviate such damage.

In a further aspect of the invention, kits for diagnosis or therapy are provided. An exemplary kit comprises a CIRL-associated molecule, such as a CIRL Protein, CIRL-encoding polynucleotide, or antibody immunologically specific for CIRL. The kits may also include a pharmaceutically acceptable carrier and/or excipient, a suitable container, and instructions for administration.

### BRIEF DESCRIPTION OF THE DRAWINGS

FIG. 1 shows a gel of separated PCR-amplified transcripts of CIRL-1, CIRL-2 and CIRL-3 from post-ischemic hippocampal cells. GAPDH was used as a control.

FIGS. 2A-2D are micrographs of hippocampal cells stained with trypan blue 16 hours after the cells were deprived of oxygen and glucose for 1 hour relative to untreated control cells (FIG. 2A). Cells deprived of oxygen and glucose for 1 hour (ischemia control cells) are shown in FIG. 2B. Ischemia control cells treated with 0.1 μM antisense oligonucleotides complementary to CIRL-1 are shown in FIG. 2C. Ischemia control cells treated with 10 μM antisense oligonucleotides complementary to CIRL-1 are shown in FIG. 2D.

FIGS. 3A and 3B are two bar graphs illustrating the results of the lactate dehydrogenase (LDH) activity assays. The data is based on four independent experiments including two hippocampal cultures and two cortical cultures. “NMDA” corresponds to complete neuronal death induced by 300 μM NMDA. FIG. 3A shows the suppression of neurodegeneration by antisense oligonucleotides complementary to CIRL-1. FIG. 3B shows the suppression of neurodegeneration by antisense oligonucleotides complementary to CIRL-3.

FIGS. 4A-4C depicts CIRL proteins and their localization in hippocampus. FIG. 4A shows a Western analysis showing the positive identification of CIRL-1 protein bands with newly developed anti-CIRL-1 antiserum. FIG. 4B depicts immunocytochemical staining using anti-CIRL-1 antiserum showing the localization of CIRL proteins in hippocampus. The CIRL immuno-positive neurons are mainly located in CA3 region. No CIRL positive neurons are found in CA1 region. FIG. 4C is a photomicrograph of higher magnification showing the squared box area in panel B. Many CA3 pyramidal neurons are labeled with anti-CIRL antiserum (arrows).

### DETAILED DESCRIPTION OF THE INVENTION

In accordance with the present invention, it has been discovered that antisense oligonucleotides complementary to CIRL-1 mRNA and CIRL-3 mRNA suppress neuronal cell death associated with hypoxia in hippocampal and cortical cell cultures. These antisense molecules may be used to advantage to treat the neurodegenerative effects of ischemic stroke by blocking neuronal cell death in patients in need thereof. The use of antisense oligonucleotides to treat ischemic stroke is extremely beneficial because this treatment may be used long after the initial period when current stroke treatments are no longer effective.

Also in accordance with this invention, methods for localizing CIRL-protein utilizing CIRL-1-specific antibodies in neuronal cells are provided. These assays demonstrate that CIRL-1 protein is primarily localized to the CA3 section of the hippocampus. This data may be used to investigate the changes in CIRL receptors in the hippocampus following transient forebrain ischemia.

The detailed description set forth below describes the preferred methods for practicing the present invention. Methods for selecting and preparing antisense oligonucleotides and antisense-encoding expression vectors are described, as well as methods for administering the antisense compositions in vivo.

To the extent that specific materials are mentioned, it is merely for purposes of illustration and is not intended to limit the invention in any way. Unless otherwise specified, general biochemical and molecular biological procedures, such as those set forth in Sambrook et al., Molecular Cloning, Cold Spring Harbor Laboratory (1989) (hereinafter “Sambrook et al.”) or Ausubel et al. (eds) Current Protocols in Molecular Biology, John Wiley & Sons (1997) (hereinafter “Ausubel et al.”) are used.

I. Definitions:

The following definitions are provided to facilitate an understanding of the present invention:

Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from-other components present during its production.

The terms “percent similarity”, “percent identity” and “percent homology” when referring to a particular sequence are used as set forth in the University of Wisconsin GCG software program.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g., chromatographic methods, agarose or polyacrylamide gel electrophoresis, HPLC analysis, and the like).

A “replicon” is any genetic element, for example, a plasmid, cosmid, bacmid, plastid, phage or virus, that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “oligonucleotide,” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The phrase “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

Appropriate conditions enabling specific hybridization of single stranded nucleic acid molecules of varying complementarity are well known in the art. For instance, one common formula for calculating the stringency conditions required to achieve hybridization between nucleic acid molecules of a specified sequence homology is set forth below (Sambrook et al., 1989):

Tₘ=81.5° C.+16.6 Log [Na+]+0.41 (% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and method of use. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by calorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell or plant.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

Amino acid residues described herein are preferred to be in the “L” isomeric form. However, residues in the “D” isomeric form may be substituted for any L-amino acid residue, provided the desired properties of the polypeptide are retained. All amino-acid residue sequences represented herein conform to the conventional left-to-right amino-terminus to carboxy-terminus orientation.

Amino acid residues are identified in the present application according to the three-letter or one-letter abbreviations in the following Table:

TABLE 1

| | 3-letter | 1-letter |
|---|---|---|
| Amino Acid | Abbreviation | Abbreviation |
| L-Alanine | Ala | A |
| L-Arginine | Arg | R |
| L-Asparagine | Asn | N |
| L-Aspartic Acid | Asp | D |
| L-Cysteine | Cys | C |
| L-Glutamine | Gln | Q |
| L-Glutamic Acid | Glu | E |
| Glycine | Gly | G |
| L-Histidine | His | H |
| L-Isoleucine | Ile | I |
| L-Leucine | Leu | L |
| L-Methionine | Met | M |
| L-Phenylalanine | Phe | F |
| L-Proline | Pro | P |
| L-Serine | Ser | S |
| L-Threonine | Thr | T |
| L-Tryptophan | Trp | W |
| L-Tyrosine | Tyr | Y |
| L-Valine | Val | V |
| L-Lysine | Lys | K |

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

By the use of the term “enriched” in reference to a polypeptide it is meant that the specific amino acid sequence constitutes a significantly higher fraction (2-5 fold) of the total of amino acid sequences present in the cells or solution of interest than in normal or diseased cells or in the cells from which the sequence was taken. This could be caused by a person by preferential reduction in the amount of other amino acid sequences present, or by a preferential increase in the amount of the specific amino acid sequence of interest, or by a combination of the two. However, it should be noted that “enriched” does not imply that there are no other amino acid sequences present, just that the relative amount of the sequence of interest has been significantly increased.

The term “significant” here is used to indicate that the level of increase is useful to the person making such an increase, and generally means an increase relative to other amino acid sequences of about at least 2 fold, more preferably at least 5 to 10 fold or even more. The term also does not imply that there are no amino acid sequences from other sources. The other source amino acid may, for example, comprise amino acid sequences encoded by a yeast or bacterial genome, or a cloning vector such as pUC19. The term is meant to cover only those situations in which a person has intervened to elevate the proportion of the desired nucleic acid.

It is also advantageous for some purposes that an amino acid sequence be in purified form. The term “purified” in reference to a polypeptide does not require absolute purity (such as a homogeneous preparation); instead, it represents an indication that the sequence is relatively purer than in the natural environment (compared to the natural level this level should be at least 2-5 fold greater, e.g., in terms of mg/ml). Purification of at least one order of magnitude, preferably two or three orders, and more preferably four or five orders of magnitude is expressly contemplated. Thus the term “substantially pure” refers to a preparation comprising at least 50-60% by weight the compound of interest (e.g., polypeptide, protein, etc.). More preferably, the preparation comprises at least 75% by weight, and most preferably 90-99% by weight, the compound of interest. Purity is measured by methods appropriate for the compound of interest (e.g. chromatographic methods, agarose or polyacrylamide gel electrophoresis, HPLC analysis, mass spectrometry and the like).

“Natural allelic variants”, “mutants” and “derivatives” of particular sequences of amino acids refer to amino acid sequences that are closely related to a particular sequence but which may possess, either naturally or by design, changes in sequence or structure. By closely related, it is meant that at least about 75%, or 80% or 85% or 90% or 95%, and often, more than 90%, or more than 95% of the amino acids of the sequence match over the defined length of the amino acid sequence referred to using a specific SEQ ID NO.

Different “variants” of CIRL-1 exist in nature. These variants may be alleles characterized by differences in the nucleotide sequences of the gene coding for the protein, or may involve different RNA processing or post-translational modifications. The skilled person can produce variants having single or multiple amino acid substitutions, deletions, additions or replacements. These variants may include inter alia: (a) variants in which one or more amino acids residues are substituted with conservative or non-conservative amino acids, (b) variants in which one or more amino acids are added to the CIRL-1 protein, (c) variants in which one or more amino acids include a substituent group, and (d) variants in which CIRL-1 is fused with another peptide or polypeptide such as a fusion partner, a protein tag or other chemical moiety, that may confer useful properties to CIRL-1, such as, for example, an epitope for an antibody, a polyhistidine sequence, a biotin moiety and the like. Other CIRL-1 proteins of the invention include variants in which amino acid residues from one species are substituted for the corresponding residue in another species, either at the conserved or non-conserved positions. In another embodiment, amino acid residues at non-conserved positions are substituted with conservative or non-conservative residues. The techniques for obtaining these variants, including genetic (suppressions, deletions, mutations, etc.), chemical, and enzymatic techniques are known to the person having ordinary skill in the art.

To the extent such allelic variations, analogues, fragments, derivatives, mutants, and modifications, including alternative nucleic acid processing forms and alternative post-translational modification forms result in derivatives of CIRL-1 that retain any of the biological properties of CIRL-1, they are included within the scope of this invention.

“Mature protein” or “mature polypeptide” shall mean a polypeptide possessing the sequence of the polypeptide after any processing events that normally occur to the polypeptide during the course of its genesis, such as proteolytic processing from a polyprotein precursor. In designating the sequence or boundaries of a mature protein, the first amino acid of the mature protein sequence is designated as amino acid residue 1. As used herein, any amino acid residues associated with a mature protein not naturally found associated with that protein that precedes amino acid 1 are designated amino acid −1, −2, −3 and so on. For recombinant expression systems, a methionine initiator codon is often utilized for purposes of efficient translation. This methionine residue in the resulting polypeptide, as used herein, would be positioned at −1 relative to the mature CIRL protein sequence.

A low molecular weight “peptide analog” or “peptidomimetic” shall mean a natural or mutant (mutated) analog of a protein, comprising a linear or discontinuous series of fragments of that protein and which may have one or more amino acids replaced with other amino acids and which has altered, enhanced or diminished biological activity when compared with the parent or nonmutated protein.

The term “biological activity” is a function or set of functions performed by a molecule in a biological context (i.e., in an organism or an in vitro surrogate or facsimile model).

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, of that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by the trained artisan, and are contemplated to be within the scope of this definition.

A “clone” or “clonal cell population” is a population of cells derived from a single cell or common ancestor by mitosis.

A “cell line” is a clone of a primary cell or cell population that is capable of stable growth in vitro for many generations.

An “antibody” or “antibody molecule” is any immunoglobulin, including antibodies and fragments thereof, that binds to a specific antigen. The term includes polyclonal, monoclonal, chimeric, and bispecific antibodies. As used herein, antibody or antibody molecule contemplates both an intact immunoglobulin molecule and an immunologically active portion of an immunoglobulin molecule such as those portions known in the art as Fab, Fab′, F(ab′)2 and F(v).

With respect to antibodies, the term “immunologically specific” refers to antibodies that bind to one or more epitopes of a protein or compound of interest, but which do not substantially recognize and bind other molecules in a sample containing a mixed population of antigenic biological molecules.

A “sample” or “patient sample” or “biological sample” generally refers to a sample which may be tested for a particular molecule, preferably a CIRL polynucleotide, polypeptide, or antibody. Samples may include but are not limited to cells, including hippocampal and cortical cells, brain or neural cells, tissue, including brain tissue, and body fluids, including cerebral-spinal fluid, blood, serum, plasma, urine, saliva, pleural fluid and the like.

II. Selection and Preparation of Antisense Oligonucleotides:

Antisense oligonucleotides targeted to any known nucleotide sequence can be prepared by oligonucleotide synthesis according to standard methods. Synthesis of oligonucleotides via phosphoramidite chemistry is preferred, since it is an efficient method for preparing oligodeoxynucleotides, as well as being adapted to many commercial oligonucleotide synthesizers.

Selection of a suitable antisense sequence depends on the knowledge of the nucleotide sequence of the target mRNA, or gene from which the mRNA is transcribed. In accordance with the present invention, the antisense molecules described herein below (SEQ ID NOS: 7 and 8) are targeted to the translation initiation sites of CIRL-1 and CIRL-3 mRNA. Although targeting to mRNA is preferred and exemplified in the description below, it will be appreciated by those skilled in the art that other forms of nucleic acid, such as pre-mRNA or genomic DNA, may also be targeted.

Synthetic antisense oligonucleotides should be of sufficient length to hybridize to the target nucleotide sequence and exert the desired effect, i.e., blocking translation of an mRNA molecule. However, it should be noted that smaller oligonucleotides are likely to be more efficiently taken up by cells in vivo, such that a greater number of antisense oligonucleotides may be delivered to the location of the target mRNA. Preferably, antisense oligonucleotides should be at least 15 nucleotides long, to achieve adequate specificity. In a preferred embodiment of the present invention, antisense molecules with 15 nucleotides in length are utilized. Optionally, antisense molecules may be 16, 17, 18, 19, 20, 21, 22, 23, 24, or 25 nucleotides in length, or may be between 25 and 35 nucleotides in length, or may be 35-50 nucleotides in length.

Small oligonucleotides such as those described above are highly susceptible to degradation by assorted nucleases. Moreover, such molecules may be unable to enter cells because of insufficient membrane permeability. For these reasons, practitioners skilled in the art generally synthesize oligonucleotides that are modified in various ways to increase stability and membrane permeability. The use of modified antisense oligonucleotides is preferred in the present invention. The term “antisense oligonucleotide analog” refers to such modified oligonucleotides, as discussed hereinbelow.

Several methods of modifying oligodeoxyribonucleotides are known in the art. For example, methylphosphonate oligonucleotide analogs may be synthesized wherein the negative charge on the internucleotide phosphate bridge is eliminated by replacing the negatively charged phosphate oxygen with a methyl group. See Uhlmann et al., Chemical Review, 90: 544-584 (1990). Another common modification, which is utilized in a preferred embodiment of the present invention, is the synthesis of oligodeoxyribonucleotide phosphorothioates. In these analogs, one of the phosphate oxygen atoms not involved in the phosphate bridge is replaced by a sulphur atom, resulting in the negative charge being distributed asymmetrically and located mainly on the sulphur atoms. When compared to unmodified oligonucleotides, oligonucleotide phosphorothioates are improved with respect to stability to nucleases, retention of solubility in water and stability to base-catalyzed hydrolysis. See Uhlmann et al., supra at 548-50; Cohen, J. S. (ed.) Oligodeoxynucleotides: Antisense Inhibitors of Gene Expression, CRC Press, Inc., Boca Raton, Fla. (1989). In a preferred embodiment of the invention, phosphorothioate-modified antisense oligonucleotides are utilized.

Other modifications of oligodeoxyribonucleotides to produce stable, membrane permeable oligonucleotide analogs are commonly known in the art. For a review of such methods, see generally, Uhlmann et al., supra, and Cohen, supra which also describe methods for synthesis of such molecules. In addition, modified oligoribonucleotides may be utilized in the present invention. However, oligodeoxyribonucleotides are preferred due to their enhanced stability, ease of manufacture and the variety of methods available for analog synthesis.

Still other modifications of the oligonucleotides may include coupling sequences that code for RNase H to the antisense oligonucleotide. This enzyme (RNase H) will then hydrolyze the hybrid formed by the oligonucleotide and the specific targeted mRNA. Alkylating derivatives of oligonucleotides and derivatives containing lipophilic groups can also be used. Alkylating derivatives form covalent bonds with the mRNA, thereby inhibiting their ability to translate proteins. Lipophilic derivatives of oligonucleotides will increase their membrane permeability, thus enhancing penetration into tissue. Besides targeting the mRNAs, other antisense molecules can target the DNA, forming triple DNA helixes (DNA triplexes). Another strategy is to administer sense DNA strands which will bind to specific regulator cis or trans active protein elements on the DNA molecule.

Deoxynucleotide dithioates (phosphorodithioate DNA) may also be utilized in this invention. These compounds which have nucleoside-OPS₂O nucleoside linkages, are phosphorus achiral, anionic and are similar to natural DNA. They form duplexes with unmodified complementary DNA. They also activate RNase H and are resistant to nucleases, making them potentially useful as therapeutic agents. One such compound has been shown to inhibit HIV-1 reverse transcriptase (Caruthers et al., INSERM/NIH Conference on Antisense Oligonucleotides and Ribonuclease H, Arcachon, France 1992).

In accordance with the present invention, antisense oligonucleotides which specifically hybridize to CIRL-1 and CIRL-3 encoding mRNA may be produced by expression of DNA sequences cloned into plasmid or retroviral vectors. Using standard methodology known to those skilled in the art, it is possible to maintain the antisense RNA-encoding DNA in any convenient cloning vector (see Ausubel et al., eds. Current Protocols in Molecular Biology, John Wiley and Sons, Inc., (1995)). In one embodiment, clones are maintained in a plasmid cloning/expression vector, such as pCEP4 (Invitrogen), which is propagated in a suitable host cell, such as hippocampal neuronal cells.

Various genetic regulatory control elements may also be incorporated into antisense encoding expression vectors to facilitate propagation in both eucaryotic and procaryotic cells. Different promoters may be utilized to drive expression of the CIRL-1 and CIRL-3 antisense sequences, the cytomegalovirus immediate early promoter being preferred as it promotes a high level of expression of downstream sequences. Polyadenylation signal sequences are also utilized to promote mRNA stability. Sequences preferred for use in the invention include, but are not limited to, bovine growth hormone polyadenylation signal sequences or thymidine kinase polyadenylation signal sequences. Antibiotic resistance markers are also included in these vectors to enable selection of transformed cells. These may include, for example, genes that confer hygromycin, neomycin or ampicillin resistance.

III. Administration of Antisense Oligonucleotides and/or Plasmid Vectors Producing Antisense Molecules:

Antisense oligonucleotides and/or antisense RNA-encoding vectors as described herein are generally administered to a patient as a pharmaceutical preparation. The term “patient” as used herein refers to human or animal subjects.

The pharmaceutical preparation comprising the antisense oligonucleotides or plasmid vectors encoding antisense RNA of the invention are conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of antisense oligonucleotides in the chosen medium will depend on the hydrophobic or hydrophilic nature of the medium, as well as the length and other properties of the antisense molecule. Solubility limits may be easily determined by one skilled in the art.

As used herein, “biologically acceptable medium” includes any and all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the antisense molecules to be administered, its use in the pharmaceutical preparation is contemplated.

Selection of a suitable pharmaceutical preparation depends upon the method of administration chosen. For example, antisense oligonucleotides may be administered by direct injection into the region of the brain containing hippocampal neuronal cells. In this instance, a pharmaceutical preparation comprises the antisense molecule dispersed in a medium that is compatible with cerebrospinal fluid. In a preferred embodiment, artificial cerebrospinal fluid (148 mM NaCl, 2.9 mM KCl, 1.6 mM MgCl₂ 6H₂O, 1.7 mM CaCl₂, 2.2 nM dextrose) is utilized, and oligonucleotides antisense to the CIRL-1 and CIRL-3 receptors are provided directly to hippocampal neuronal cells. In another preferred embodiment, the antisense oligonucleotides are administered by direct injection into the hippocampus or cortical regions of the brain.

Oligonucleotides antisense to CIRL-1 and CIRL-3 mRNAs may also be administered parenterally by intravenous injection into the blood stream, or by subcutaneous, intramuscular or intraperitoneal injection. Pharmaceutical preparations for parenteral injection are commonly known in the art. If parenteral injection is selected as a method for administering the antisense oligonucleotides, steps must be taken to ensure that sufficient amounts of the molecules reach their target cells to exert the desired biological effect. The lipophilicity of the antisense molecules, or the pharmaceutical preparation in which they are delivered may have to be increased so that the molecules can cross the blood-brain barrier to arrive at their target locations. Furthermore, the antisense molecules may have to be delivered in a cell-targeted carrier so that sufficient numbers of molecules will reach the target cells. Methods for increasing the lipophilicity of a molecule are known in the art, and include the addition of lipophilic groups to the antisense oligonucleotides. Phosphorothioate or methylphosphonate oligonucleotide analogs become widely dispersed in living tissues following intravenous injection.

For example, experiments in mice, which provided a detailed analysis of the pharmacokinetics, biodistribution and stability of oligonucleotide phosphorothioates showed a widespread distribution of phosphorothioate-modified oligodeoxynucleotides in most tissues for up to 48 hours. Significant amounts were found in brain following intraperitoneal or intravenous administration. Agrawal et al., Proc. Natl. Acad. Sci. USA, 88: 7595-99 (1991). In another study, methylphosphonate oligonucleotides were injected into mouse tail veins and found to achieve a reasonably uniform distribution in mouse tissue. See Uhlmann et al., supra at 577, citing Miller et al., Anti-Cancer Drug Design, 2: 117 (1987).

Several techniques have been used to increase the stability, cellular uptake and biodistribution of oligonucleotides. Antisense oligonucleotides of the present invention may be encapsulated in a lipophilic, targeted carrier, such as a liposome. One technique employs a carrier for the oligonucleotide comprising a liposomal preparation containing the cationic lipid N-[1-(2,3-dioleyloxy)propyl]-N,N,N-trimethyl ammonium chloride (D OT MA; lipofectin). This has been found to increase by about 1000 fold the potency of the antisense oligonucleotide ISIS 1570, which hybridizes to the AUG translation initiation codon of human intracellular adhesion molecule-1. Bennett et al., Mol Pharmacol., 41: 1023-1033 (1992). Phosphorothioates have also been particularly useful for increasing the biodistribution and stability of oligodeoxynucleotides in mice as described above. Loading phosphorothioate oligonucleotides into liposomes, particularly pH sensitive liposomes, to increase their cellular uptake has also been used with some success. Loke et al., Curr. Topics Microbiol. Immunol., 141: 282-289 (1988); Connor and Huang, Cancer Res., 46: 3431-3435 (1986).

Both the oligonucleotides and vectors of the present invention may be complexed to liposomes. To further facilitate targeting of the CIRL-1 and CIRL-3 encoding mRNA molecules, liposomes may be “studded” with antibodies specific for certain regions of the brain (Leserman et al., (1980) Nature 288:604). In a preferred embodiment, cationic liposomes are complexed with (1) CIRL-1 or CIRL-3 mRNA antisense oligonucleotide or vector encoding antisense RNA; and (2) antibodies specific for the hippocampus. Vector containing antibody-studded-liposome complexes are expected not only to be targeted and specifically expressed in the hippocampal region of the brain, but also to be expressed for a longer duration than that observed with antisense oligonucleotide delivery alone.

Additional means by which antisense oligonucleotides may be administered include oral administration and intranasal or ophthalmic administration.

The pharmaceutical preparation is formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of antisense oligonucleotides directed to CIRL-1 and CIRL-3 encoding mRNA may be determined by evaluating the toxicity of the antisense oligonucleotides in animal models. Various concentrations of antisense pharmaceutical preparations may be administered to mice affected by ischemic stroke, and the minimal and maximal dosages may be determined based on the results of significant reduction in neurodegeneration as a result of the antisense oligonucleotide treatment. Appropriate dosage unit may also be determined by assessing the efficacy of the antisense oligonucleotide treatment in combination with other standard stroke treatments, such as tPA. The dosage units of antisense oligonucleotide may be determined individually or in combination with tPA.

The pharmaceutical preparation comprising the antisense oligonucleotides may be administered at appropriate intervals, for example, twice a day until the stroke symptoms are reduced or alleviated, after which the dosage may be reduced to a maintenance level. The appropriate interval in a particular case would normally depend on the condition of the patient. Additionally, the pharmaceutical preparation of the invention may include additional pharmacological agents which are useful for treating neurodegeneration-related disorders.

While the above discussion refers to the delivery of antisense oligonucleotides, it will be apparent to those skilled in the art that the methods described would also be suitable for the delivery of the vector constructs encoding CIRL-1 and CIRL-3 mRNA-specific antisense oligonucleotides.

IV. CIRL-1 Antibodies and Methods of Making the Same

The present invention also provides methods of making and methods of using antibodies capable of immunospecifically binding to CIRL-1 protein or fragments thereof. Polyclonal antibodies directed toward CIRL-1 protein may be prepared according to standard methods. In a preferred embodiment, monoclonal antibodies are prepared, which react immunospecifically with the various epitopes of the CIRL-1 protein. Monoclonal antibodies have been prepared according to general methods of Köhler and Milstein, following standard protocols.

Purified CIRL-1, or fragments thereof, may be used to produce polyclonal or monoclonal antibodies which also may serve as sensitive detection reagents for the presence and accumulation of CIRL-1 protein in mammalian brain tissue. Recombinant techniques enable expression of fusion proteins containing part or all of the CIRL-1 protein. The full length protein or fragments of the protein may be used to advantage to generate an array of monoclonal antibodies specific for various epitopes of the protein, thereby providing even greater sensitivity for detection of the protein in cells.

Antibodies according to the present invention may be modified in a number of ways. Indeed the term “antibody” should be construed as covering any binding substance having a binding domain with the required specificity. Thus, the invention covers antibody fragments, derivatives, functional equivalents, and homologues of antibodies, including synthetic molecules and molecules whose shape mimics that of an antibody enabling it to bind an antigen or epitope.

Exemplary antibody fragments, capable of binding an antigen or other binding partner, are Fab fragment consisting of the VL, VH, Cl and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region. Single chain Fv fragments are also included.

Humanized antibodies in which CDRs from a non-human source are grafted onto human framework regions, typically with alteration of some of the framework amino acid residues, to provide antibodies which are less immunogenic than the parent non-human antibodies, are also included within the present invention.

Polyclonal or monoclonal antibodies that immunospecifically interact with CIRL-1 protein can be utilized for identifying and purifying CIRL-1. For example, antibodies may be utilized for affinity separation of proteins with which they immunospecifically interact. Antibodies may also be used to immunoprecipitate proteins from a sample containing a mixture of proteins and other biological molecules. Other uses of anti-CIRL-1 antibodies are described below.

V. Methods of Using CIRL-1 Polynucleotides, Polypeptides, and Antibodies for Screening and Diagnostic Assays

CIRL-1-antisense nucleic acids may be used for a variety of purposes in accordance with the present invention. Methods in which CIRL-1-antisense nucleic acids may be utilized include, but are not limited to: (1) down regulation of CIRL expression; (2) Southern hybridization; and (3) northern hybridization.

Polyclonal or monoclonal antibodies immunologically specific for CIRL-1 may be used in a variety of assays designed to detect and quantitate the protein. Such assays include, but are not limited to: (1) flow cytometric analysis; (2) immunochemical localization of CIRL-1 in brain cells; and (3) immunoblot analysis (e.g., dot blot, Western blot) of extracts from various cells. Additionally, as described above, anti-CIRL-1 can be used for purification of CIRL 1 (e.g., affinity column purification, immunoprecipitation).

VI. Kits and Articles of Manufacture

Any of the aforementioned products are methods can be incorporated into a kit which may contain a polynucleotide, an oligonucleotide, a polypeptide, a peptide, an antibody, a label, marker, or reporter, a pharmaceutically acceptable carrier, a physiologically acceptable carrier, instructions for use, a container, a vessel for administration, an assay substrate, or any combination thereof.

Further details regarding the practice of this invention are set forth in the following examples, which are provided for illustrative purposes only and are in no way intended to limit the invention.

### EXAMPLE I

### CIRL mRNA Expression in CA1 and CA3 Neurons

The expression of calcium-independent receptors for α-latrotoxin (CIRL) in CA1 neurons and CA3 neurons from hippocampus before and after ischemic attack was analyzed to further uncover the molecular mechanism(s) which regulate neurodegeneration after cerebral ischemic attack.

I. Materials and Methods:

To search for any alteration in mRNA expression of CIRLs in CA1 and CA3 neurons in hippocampus, ischemic insults were induced using a four-vessel occlusion method as described previously (8, 10). NIH guidelines for the care and use of laboratory animals were strictly followed. Briefly, adult male Wistar rats were starved overnight to produce uniform blood glucose levels. The rats were anesthetized with 1-2% halothane mixed with 33% O₂ and 66% N₂. The vertebral arteries were then electrocauterized and the common carotid arteries were occluded to induce ischemic depolarization for approximately 14 minutes. Brain temperature was maintained at 37° C. during ischemia. Hippocampal tissues were collected from CA1 and CA3 regions at 1, 6, 12, and 36 hours after reperfusion. Total RNA was isolated from the collected tissue using RNeasy mini kits (Qiagen, 9), and reverse transcription/polymerase chain reaction (PCR) was conducted using pairs of PCR primers specific for CIRL-1, CIRL-2, and CIRL-3 mRNA. The PCR primers are provided in Table 1.

TABLE 1

PCR PRIMERS

| | |
|---|---|
| CIRL-1 | |
| 5′-CCTCAGCCATCGCGGCTAACGCC-3′ | (SEQ ID NO: 1) |
| 5′-TGAAGCCCACAGACTCTGCAATG-3′ | (SEQ ID NO: 2) |
| CIRL-2 | |
| 5′-CTGATCCATGTCCCGGAACTT-3′ | (SEQ ID NO: 3) |
| 5′-CGTCCACTCGGTTTGGAAGTT-3′ | (SEQ ID NO: 4) |
| CIRL-3 | |
| 5′-GACATCTTCTTCAGCAGCCAG-3′ | (SEQ ID NO: 5) |
| 5′-CACTGCACACTGGGTTCTGTT-3′ | (SEQ ID NO: 6) |

II. Results:

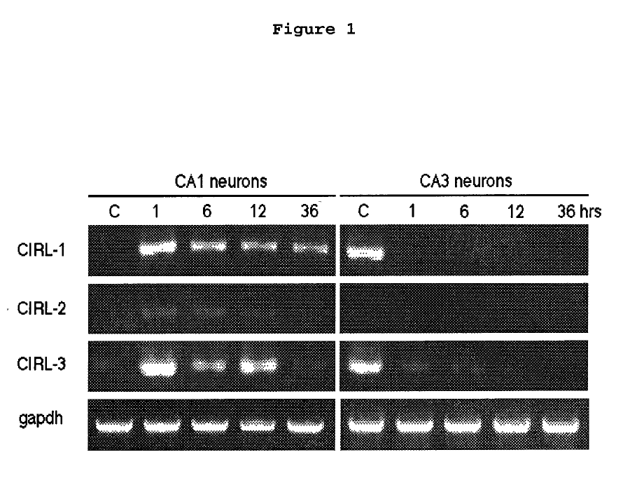

The mRNA expression levels of CIRL-1 and CIRL-3 differed distinctively in CA1 neurons and CA3 neurons before and after ischemic insult to hippocampal tissue. Prior to ischemic treatment, both CIRL-1 mRNA and CIRL-3 mRNA were undetectable in CA1 neurons, but were clearly expressed in CA3 neurons (See FIG. 1). Interestingly, in post-ischemic hippocampal tissue, CIRL-1 and CIRL-3 mRNAs were expressed in CA1 neurons, but were undetectable in CA3 neurons. Since CA1 neurons are more susceptible to neuronal cell death during ischemia than CA3 neurons, these results suggest that CIRL-1 and CIRL-3 may play a role in the neurodegeneration of CA1 neurons. Alterations in mRNA expression of CIRL-2 were not detected in either CA1 or CA3 neurons.

### EXAMPLE 2

### Antisense Oligonucleotide Treatment Blocks Neurodegeneration

Based on the differential mRNA expression of CIRL-1 and CIRL-3 in CA1 and CA3 neurons in response to transient ischemia, the potential role of CIRLs in neurodegeneration was investigated by blocking the translation of CIRL-1 or CIRL-3 mRNAs using antisense oligonucleotides complementary to CIRL-1 and CIRL-3 mRNA (1). Specifically, the transient transfer of antisense oligonucleotides in vitro into hippocampal or cortical neurons was examined to determine whether the antisense treatment would suppress cellular death of neurons cultured in media lacking oxygen and glucose.

I. Materials and Methods:

Hippocampal tissue and cortical tissue were harvested from fetal Wistar rats at 17-18 day gestation and the dissociated cells were placed in a 24 well-culture plate containing Eagle's minimal essential medium supplemented with 20 mM glucose, 10% fetal bovine serum, and antibiotics (3). Cultures were used for in vitro experiments after 12 days. To simulate ischemia in vitro, the cultured cells were incubated in deoxygenated, glucose-free Earle's balanced salt solution in anaerobic conditions for 1 hour. For blocking translation of CIRL-1 and CIRL-3 mRNAs, the cultured cells were incubated with 0.1 μM, 1 μM or 10 μM phosphorothioate-modified antisense oligonucleotides complementary to CIRL-1 (5′-GGGCCATGGCGAAGG-3′; SEQ ID NO: 7) and CIRL-3 (5′-GACACATGGCTGTGT-3′; SEQ ID NO: 8) (Ana-Gen Technologies, Inc.). The cultured cells were incubated with these antisense oligonucleotides for 3 hours in a pre-ischemic/hypoxic period, followed by ischemic/hypoxia treatment for 1 hour, and then for 16 to 48 hours in the post-ischemic/hypoxic period. Sense strand oligonucleotides were used as a control. All of the oligonucleotides described herein were HPLC-purified prior to their use.

To assess neuronal cell death in the hippocampal and cortical tissue cultures, cellular morphology was monitored by staining damaged cells with 0.4% trypan blue (Life Technologies, 4). Lactate dehydrogenase (LDH) activity was also measured using an in vitro toxicology kit (Sigma, 2). In the LDH assay, high LDH activity resulted in low spectrophotometric readings for LDH-activity-involved dye molecules and the control for complete neuronal cell death was created by exposing the control cultures to 300 μM -methyl-D-aspartate (NMDA).

II. Results:

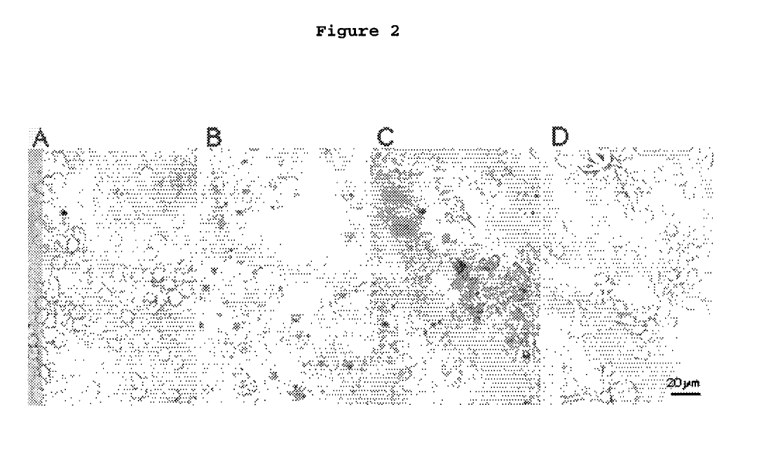

Administration of the antisense oligonucleotides of the invention clearly suppressed the adverse affects of ischemia as evidenced by analysis of cellular morphology at 16 and 24 hours following deprivation of oxygen and glucose. Unlike normal control cultures, deprivation of oxygen and glucose for 1 hour increased the number of trypan-blue stained ischemia control cells (See FIGS. 2A and 2B). An average number of trypan-blue stained hippocampal cells in a 1 mm×1 mm field of view was approximately 3±2 for the normal control and 52±6 for the ischemia/hypoxia-treated cells, respectively. The neuronal cell cultures incubated with antisense DNA for CIRL-1 mRNA had vastly reduced numbers of stained cells as compared to the ischemia control cells not treated with antisense oligonucleotides (FIGS. 2C and 2D). These observations were consistent with duplicate experiments for both hippocampal and cortical cultures as the antisense oligonucleotides complementary to CIRL-1 mRNA and CIRL-3 mRNA were equally effective in suppressing neuronal degeneration in hippocampal and cortical neurons in vitro (data not shown).

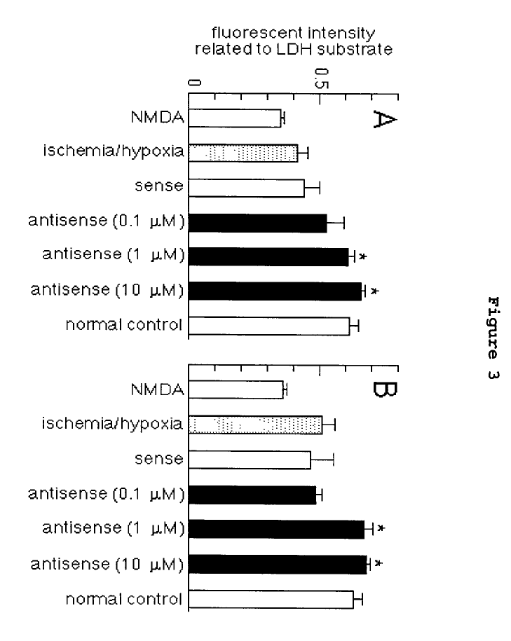

The results of the LDH assay for neuronal cell death 16 hours after ischemic treatment also demonstrated the suppressive effects of the antisense oligonucleotide treatment on neurodegeneration in vitro in hippocampal and cortical cultures. The suppressive effects occurred in an oligonucleotide-concentration dependent manner (See FIG. 3). LDH activity levels measured from the neuronal cell cultures treated with either 10 μM CIRL-1 antisense or 10 μM CIRL-3 antisense oligonucleotides were essentially identical to the levels of LDH activity measured from the control cultures. Similarly, the LDH activity levels measured from neuronal cells treated with 10 μM sense strand oligonucleotides were nearly identical to the LDH activity levels measured from neuronal cells that were not treated with either sense or antisense oligonucleotides.

III. Discussion:

Based on the results described above in combination with the results previously described for neurexin calcium-dependent receptors, it appears that at least two families of a-latrotoxin receptors, the neurexins and CIRLs, have altered mRNA expression levels in post-ischemic neuronal cells, and their expression in response to ischemia differs between CA1 and CA3 neurons. The data also implicate receptors for α-latrotoxin may have a significant role in the molecular mechanism(s) that cause cellular death of CA1 neurons in response to ischemia and hypoxia. Three mechanisms for α-latrotoxin-mediated signal transduction have already been proposed (5); however, further studies are necessary to elucidate the CIRL-mediated molecular pathway that controls neuronal cell death.

In addition, this study demonstrates that both CIRL-1 and CIRL-3 transcripts were differentially expressed in CA1 and CA3 hippocampal neurons in response to transient global ischemia in vivo, and that antisense oligonucleotides complementary to CIRL-1 and CIRL-3 mRNA suppressed neurodegeneration in response to ischemia/hypoxia in vitro. Hence, these antisense oligonucleotide molecules may be used to advantage as therapeutic agents for the treatment of ischemic stroke and its ensuing complications.

### EXAMPLE III

### Identifying the CIRL Protein and it's Localization in the Brain with anti-CIRL-1 Antibodies

Anti-CIRL-1 antibodies were utilized to localize CIRL-1 expression in the brain. It was determined that CIRL-1 is primarily expressed in the CA3 section of the hippocampus.

I. Materials and Methods

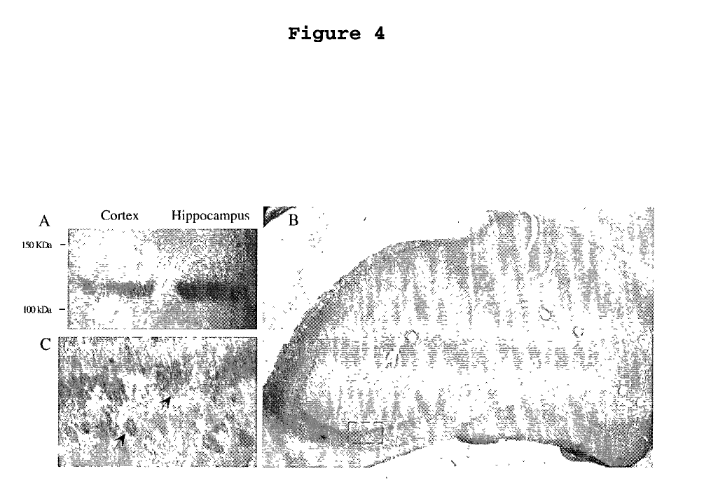

Specific antibodies against CIRL-1 protein were recently developed from rabbit. (Rockland Gilbertsville, Pa.). Immuno-detection of CIRL-1 protein with CIRL-1 specific antiserum was performed. Cortical and hippocampal tissues of a rat brain were solubilized in a lysis buffer (0.1% SDS, 0.5% sodium deoxycholate, 1% Nonidet P-40, 50 mM Tris-HCl, pH 8.0, and 150 mM NaCl). Approximately 10 μg of the protein extract from each tissue was separated by electrophoresis on a 10% SDS PAGE gel, and the gel was blotted onto nitrocellulose. A protein band corresponding to 120 kDa CIRL-1 was detected using the primary antibodies specific to CIRL-1 and the secondary antibodies conjugated to horseradish peroxidase (ECL Western blotting analysis system, Amersham) (FIG. 4A).

The localization of CIRL receptors in hippocampus was investigated using immunocytochemical techniques. Coronal sections of 50 μm thickness were cut and incubated with anti-CIRL-1 serum (1:4,000) for 36 h at 8° C. followed by biotinylated secondary antibodies and then HRP conjugated Avidin-Biotin Complex. As shown in FIG. 4B, the CIRL positive neurons were mainly located in CA3 region, and no immunopositive neurons were found in CA1 region. Higher magnification picture (FIG. 4C) indicated that the cell bodies of most CA3 pyramidal neurons were labeled by CIRL-1 antibodies (arrows).

II. Results

These results demonstrated the specificity of anti-CIRL antiserum and the localization pattern of CIRL proteins in hippocampus. The distribution of CIRL positive neurons in hippocampus of intact animals coincides with the CIRL mRNA expression pattern. These data provide a basis for investigating the changes of CIRL receptors in hippocampus following transient forebrain ischemia.

### REFERENCES

1. Bennett, C. F., and Cowsert, L. M., Antisense oligonucleotides as a tool for gene functionalization and target validation. Biochim. Biophys. Acta 1489 (1999) 19-30.

2. Decker, T., and Lohmann-Matthes, M. L., A quick and simple method for the quantitation of lactate dehydrogenase release in measurements of cellular cytotoxicity and tumor necrosis factor (TNF) activity. J. Immunol. Methods 15 (1988) 61-69.

3. Goldberb, M. P., Strasser, U., and Dugan, L. L., Techniques for assessing neuroprotective drugs in vitro. Neuroprotective Agents and Cerebral Ischaemia 69-93.

4. Goldberg, M. P., and Choi, D. W., Combined oxygen and glucose deprivation in cortical cell culture: calcium-dependent and calcium-independent mechanisms of neuronal injury. J. Neurosci. 13 (1993) 3510-3524.

5. Henkel, A. W., and Sankaranarayanan, S., Mechanisms of α-latrotoxin action. Cell Tissue Res. (1999) 229-233.

6. Lee, J. M., Zipfel, G. J., and Choi, D. W., The changing landscape of ischaemic brain injury mechanisms. Nature 399 Supp. (1999) A7-A14.

7. Mitani, A., Andou, Y., and Kataoka, K., Selective vulnerability of hippocampal CA1 neurons cannot be explained in terms of an increase in glutamate concentration during ischemia in the gerbil: Brain microdialysis study. Neurosci. 48 (1992) 307-313.

8. Pulsinelli, W. A., and Brierley, J., A new model of bilateral hemispheric ischemia in the unanesthetized rat. Stroke 10 (1979) 267-272.

9. Sun, H. B., Yokota, H., Chi, X. X., and Xu, Z. C., Differential expression of neurexin mRNA in CA1 and CA3 hippocampal neurons in response to ischemic insult. Mol. Brain Res. 84 (2000) 146-149.

10. Xu, Z. C., Gao, T. M., and Ren, Y., Neurophysiological changes associated with selective neuronal damage in hippocampus following transient forebrain ischemia. Biol. Signals Recept. 8 (1999) 294-308.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An antisense molecule targeted to a nucleic acid molecule encoding a calcium-independent receptor for α-latrotoxin (CIRL), wherein said antisense molecule specifically hybridizes with said nucleic acid molecule encoding CIRL and inhibiting the expression of CIRL.

2. The antisense molecule of claim 1 which is an antisense oligonucleotide.

3. The antisense oligonucleotide of claim 2 selected from the group consisting of SEQ ID NO: 7 and SEQ ID NO: 8.

4. The antisense oligonucleotide of claim 3 having the sequence of SEQ ID NO:7, which is 5′-GGGCCATGGCGAAGG-3′.

5. The antisense oligonucleotide of claim 3 having the sequence of SEQ ID NO: 8, which is 5′-GACACATGGCTGTGT-3′.

6. The antisense oligonucleotide of claim 3, wherein said antisense oligonucleotide blocks neurodegeneration of hippocampal neuron cells caused by ischemia.

7. The antisense oligonucleotide of claim 3, wherein said antisense oligonucleotide comprises at least one modified internucleoside linkage.

8. The antisense oligonucleotide of claim 7, wherein said modified internucleoside linkage is a phosphorothioate linkage.

9. A method of inhibiting the expression of CIRL in human cells or tissues in vitro comprising contacting said cells or tissues in vitro with the antisense molecule of claim 1 so that expression of CIRL is inhibited.

10. A method for inhibiting the expression of CIRL in human cells, said method comprising:
a) providing an antisense oligonucleotide of claim 3 which hybridizes to an expression-controlling sequence of a nucleic acid molecule that encodes CIRL; and
b) administering said antisense oligonucleotide to said humans cells under conditions causing said antisense oligonucleotide to enter said human cells expressing CIRL and bind specifically to the expression-controlling sequence of said nucleic acid molecule encoding CIRL and in an amount sufficient to inhibit expression of said CIRL.

11. A method of claim 10, wherein said human cells are hippocampal CA1 neuronal cells.

12. A method according to claim 11, wherein administration of said antisense oligonucleotide blocks neurodegeneration of said hippocampal CA1 neuronal cells.

13. A method according to claim 10, wherein said antisense oligonucleotide is an antisense oligonucleotide analog.

14. The antisense oligonucleotide of claim 2, wherein said antisense oligonucleotide is encoded by DNA.

15. A vector comprising the DNA which encodes the antisense oligonucleotide of claim 14.

16. A method of treatment for ischemic stroke, said method comprising delivery of an antisense oligonucleotide of claim 3 which enters hippocampal cells and binds specifically to a nucleic acid molecule encoding a CIRL in an amount sufficient to inhibit expression of said CIRL.

17. A pharmaceutical preparation for treating ischemic stroke, comprising an antisense oligonucleotide analog which enters hippocampal cells expressing CIRLs and binds specifically to a nucleic acid molecule encoding a CIRL, said antisense oligonucleotide being present in a biologically compatible medium.

18. A pharmaceutical preparation according to claim 17, wherein said antisense oligonucleotide comprises the sequence of SEQ ID NO:7, which is 5′-GGGCCATGGCGAAGG-3′.

19. A pharmaceutical preparation according to claim 17, wherein said antisense oligonucleotide comprises the sequence of SEQ ID NO:8, which is 5′-GACACATGGCTGTGT-3′.

20. A pharmaceutical preparation according to claim 17, which further comprises at least one targeting agent for improving delivery of said antisense oligonucleotide to said hippocampal cells.

21. An antibody which specifically binds to a calcium-independent receptor for α-latrotoxin (CIRL).

22. A method of identifying a CIRL-1 protein localization in the brain comprising
a) providing an antibody which specifically binds to a calcium-independent receptor for α-latrotoxin (CIRL);
b) contacting a brain tissue sample with said antibody, said antibody further comprising a detectable label;
c) detecting said detectably-labeled antibody, thereby localizing said CIRL-1 protein in said brain tissue.

23. The method of claim 22 further comprising;
d) determining whether CIRL protein expression levels are altered in response to ischemic damage.


SEQUENCE LISTING (8 sequences)

SEQ ID NO: 1 - DNA, 23 bp, Artificial Sequence
  primer
cctcagccatcgcggctaacgcc

SEQ ID NO: 2 - DNA, 23 bp, Artificial Sequence
  primer
tgaagcccacagactctgcaatg

SEQ ID NO: 3 - DNA, 21 bp, Artificial Sequence
  primer
ctgatccatgtcccggaactt

SEQ ID NO: 4 - DNA, 21 bp, Artificial Sequence
  primer
cgtccactcggtttggaagtt

SEQ ID NO: 5 - DNA, 21 bp, Artificial Sequence
  primer
gacatcttcttcagcagccag

SEQ ID NO: 6 - DNA, 21 bp, Artificial Sequence
  primer
cactgcacactgggttctgtt

SEQ ID NO: 7 - DNA, 15 bp, Artificial Sequence
  oligonucleotide
gggccatggcgaagg

SEQ ID NO: 8 - DNA, 15 bp, Artificial Sequence
  oligonucleotide
gacacatggctgtgt